(19) United States
(12) Patent Application Publication
(10) Pub. No.: US 2026/0102380 A1
(43) Pub. Date: Apr. 16, 2026

(54) Synthesis and Characterization of Cyclic Hydrocarbon Esters of Everolimus

(71) Applicant: BIOTRONIK AG, Buelach (CH)

(72) Inventor: Ronald E. Betts, La Jolla, CA (US)

(21) Appl. No.: 19/116,625

(22) PCT Filed: Aug. 29, 2023
(86) PCT No.: PCT/EP2023/073586

§ 371 Date: Mar. 28, 2025

Related U.S. Application Data
(60) Provisional application No. 63/412,248, filed on Sep. 30, 2022.

(30) Foreign Application Priority Data
Dec. 1, 2022 (EP) 22210799.7

(51) Int. Cl. A61K 31/436; A61K 9/08; A61K 47/10; C07D 498/18

(52) U.S. Cl. CPC A61K 31/436; A61K 9/08; A61K 47/10; C07D 498/18

(57) ABSTRACT

Generally, the field of synthesis, formulations and perivascular treatment methods involving cyclic ester derivatives of everolimus. More specifically, a method of treating open vascular surgical manipulations comprising administering an effective amount of the rapamycin 40-O-cyclic hydrocarbon ester compound, preferably by direct perivascular application, wherein the compound is preferably formulated to allow for direct perivascular application directly to graft vessels upon initiation of surgery.

### FIELD OF THE INVENTION

This application relates generally to the field of synthesis, formulations and perivascular treatment methods involving cyclic ester derivatives of everolimus.

### BACKGROUND OF THE INVENTION

Occlusive vascular diseases are a leading cause of mortality and morbidity worldwide. Recently use of drug eluting stents and drug coated balloons for minimally invasive percutaneous endovascular interventions have reduced the frequency of stenotic situations. However, unlike the technical advances of using drug eluting stents and drug coated balloons, there are no approved clinical options for preventing surgery induced intimal hyperplasia in “open” vasculature intervention. (Chen, et al., Biomacromolecules, (18)2205-2213, 2017) Open surgery is still the only solution for cases considered unsuitable for the percutaneous approach, including coronary bypass surgery, carotid endarterectomy, autologous arteriovenous fistula grafting procedures for hemodialysis patients, stenosis after kidney transplant and for treating various types of complications following endovascular surgery.

Limited flow stenotic conditions continue to be a major cause of surgical failure in these cases. A further complication in such surgically manipulated vessels is the induced occurrence of angiosarcoma or cutaneous squamous cell carcinoma at the surgery site. (Oskrochi, et al., Eur J Vasc Endovasc Surg, (51)127-133, 2015; Jennings, et al., Cancer, (62)11:2436-2444, 1988; Costa, et al., Case Rept Transplant, 2426859, 2017)

There are approximately 400,000 open vascular procedures annually in the United States (Jim et al., J Vasc Surg (55)5:1394-1400, 2012) Open procedures result in up to 50% occluding (restenosis) depending on the type of surgical intervention. An example of open intervention is that required for hemodialysis treatment (HD). HD is driven by chronic kidney disease that continues to be a global public health issue. In the United States approximately 750,000 patients per year are affected by end stage renal disease (ESRD) which is increasing by 5% per year. (https://pharm.ucsf.edu/kidney/need/statistics, accessed May 8, 2019) ESRD is total and permanent kidney failure and is treated with a kidney transplant or dialysis. In 2016, 71% of all U.S. ESRD cases were receiving HD therapy. (https://www.kidney.org/news/newsroom/factsheets/End-Stage-Renal-Disease-in-the-US, accessed May 8, 2019) The process of HD requires frequent blood access and the arteriovenous fistula (AVF) created through open surgery has emerged as the preferred blood access method. The AVF utilizes an artery surgically connected directly to a vein, usually in the arm which allows blood to be withdrawn and returned on a regular basis. The AVF is not without significant restenotic and graft failure problems in both the U.S.A. and Europe. (Gallieni, et al., Semin Intervent Radiol (26)96-105, 2009) The current treatment of such flow limited vessels is usually by use of balloon angioplasty, stenting or new graft formation. These retreatment procedures can be compromised because of the complicated nature of the surgically modified vessels which can include unnatural orientations and small sizes. Additionally, besides patient inconveniences, there are increased patient surgical risk factors, frequent need for blood thinning drugs over a period of time and increased costs.

Initial attempts at treatment means to mitigate or reduce vessel restenosis focused primarily on two areas: placement of perivascular (surrounding the outside) drug-free medical devices including steel alloy meshes, nitinol meshes and Dacron® sheaths to hold or reinforce the graft, or the systemic delivery of various vasoactive compounds. In the case of medical devices, the surgical procedures were prone to induce increased vessel injury and results demonstrated low or contradictory efficacy. (Mylonaki, et al., Biomaterials (128)56-68, 2017; Rescigno, et al., Thorac Cardiovasc Surg, (63)4:292-297, 2015) While systemic drug delivery resulted in only modest promise, there was still the potential for adverse systemic toxicity. (Schömig, et al., Circulation (112)2759-2761, 2005; Davies, et al., Eur J Vasc Endovasc Surg (42)519-529, 2011)

To overcome negative systemic drug effects, the direct targeted application of neat pharmaceutical agents has been attempted. (Chaudhary, et al., J Control Release, (233)174-180, 2016) These direct drug applications have also resulted in poor overall outcome due to fast drug washout and clearance and/or drug migration when used without a mechanism to retain the drug at the site where needed. Resulting attempts to overcome washout effects necessitated much higher drug loading and possible local toxicities. Together those findings suggested that a drug and drug carrier of some type was needed to facilitate both placement and retention of drug at the surgical site to provide control and sustained drug delivery over the course of treatment.

Ensuing development work has investigated a variety of drug-carrying, perivascular systems envisioned to provide IH treatment at surgical sites. A suggested classification of these systems has recently been provided: Gel, Gel+particles, Mesh, Sheath, Cuff, Wrap and Matrix. These authors compared the properties of each system as well. (Mylonaki, op. cit.) This comparison took into account the case of administration, possibility of repeat administration, site specific localization, mechanical support and sustaining the drug release. The concluding opinion of these authors was that a system optimizing all requirements has not yet been developed.

Even now as then commented by Seedial, there are currently no approved techniques or drug delivery devices for reducing restenosis after open surgical revascularizations (Seedial, S. M., Kent, K. C., and et. al. “Local drug delivery to prevent restenosis” Journal of Vascular Surgery, Vol. 57 pp. 1403-1414, 2013). Applying a drug extraluminally appears promising since drug applied outside of the vessel has been shown to diffuse into the blood vessel wall (Lovich, M. A., Brown, L., and Edelman, E. R. “Drug Clearance and Arterial Uptake After Local Perivascular Delivery to the Rat Carotid Artery” J. Am. Coll. Cardiol., Vol. 29 pp. 1645-50, 1997). It has been argued that extraluminal injury from surgical revascularization promotes signal cascade that activates migration of myofibroblasts into the neointima resulting in intimal hyperplasia; drug applied extraluminally would reduce the migration of myofibroblasts and lessen restenosis (Siow, R. C. M., and Churchman, A. T. “Adventitial growth factor signaling and vascular remodeling: potential of perivascular gene transfer from the outside-in” Cardiovasc. Re., Vol. 75 pp. 659-68, 2007).

Thus, there is a need in the state of the art to improve upon the above described deficiencies in the field of improved drug delivery at the specific site of surgery. Loss of vascular patency after open surgery continues to be a world-wide problem. There continues to be a lack of an approved drug, device or drug/device specific to this occlusive disease. Treatment of this condition is a large unmet need which requires a novel solution to remedy the aforementioned problems in the relevant art.

### SUMMARY OF THE INVENTION

The present invention provides for a rapamycin 40-O-cyclic hydrocarbon ester compound having the structure:

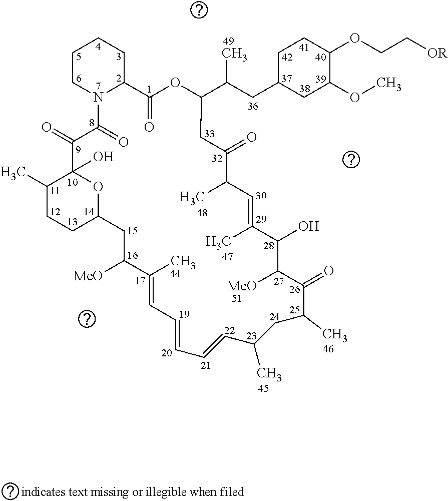

where R is a saturated cyclic hydrocarbon substituent. Preferably, R has one of the following structures:

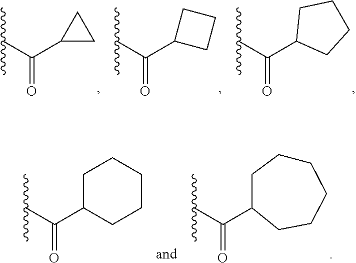

Surprisingly, it was found that these compounds having a cyclic hydrocarbon substituent at the C40 ethoxyether position show extraordinary useful behavior when administered in a perivascular fashion. Inventors have found that the compounds as described above exhibit an unusually slow drug elution from vessel tissue, indicating a very slow drug wash-out in perivascular treatment. Thereby, a very sustained perivascular drug release to the tissue is facilitated addressing the current problem of preventing surgery induced intimal hyperplasia in “open” vasculature intervention.

In another aspect, the present invention provides for a method of treating open vascular surgical manipulations comprising administering an effective amount of the rapamycin 40-O-cyclic hydrocarbon ester compound described above, preferably by direct perivascular application, wherein the compound is preferably formulated to allow for direct perivascular application directly to graft vessels upon initiation of surgery. Preferably, the compound is stabile when administered to a coronary artery or to a peripheral artery. Most preferably, the compound, once administered, results in superior vessel retention and reduced vessel diffusion. Preferably, open vascular surgical manipulations include coronary bypass surgery, carotid endarterectomy, arteriovenous fistula grafting (hemodialysis), stenosis after kidney transplant and endovascular surgery.

In yet another aspect, the present invention provides for a method of treating a vascular injury comprising providing the compound described above, wherein the compound is dissolved in at least one solvent, thereby creating a drug formulation, wherein the drug formulation is applied directly to at least one tissue within the vascular injury site. Preferably, the at least one solvent comprises or consists of an alcohol. In that the alcohol acts as a solubilizer.

In another aspect, the present invention is directed to an implant carrying an effective amount of the rapamycin 40-O-cyclic hydrocarbon ester compound described above. Preferably, such implant is a patch, graft, suture material.

In another aspect, the present invention is directed to a formulation including the rapamycin 40-O-cyclic hydrocarbon ester compounds as described herein and at least one solvent, wherein the solvent consists of or comprises at least one solubilizer or a mixture of solubilizers. The solubilizer can be selected from the group consisting of alcohols such as ethanol, propanol, butanol, benzyl alcohol, glycerol, polytheylene glycol and propylene glycol, dimethyl sulfoxide, dimethylformamide, propylene benzyl benzoate, glycol monolaurate, Labrasol®, Kolliphor®, and amylene hydrate.

### BRIEF DESCRIPTION OF THE DRAWINGS

The disclosed subject matter contained herein is best described in conjunction with the accompanying drawings, in which:

FIG. 1 shows the main structure of everolimus and the cyclic hydrocarbon esters thereof, as defined by the R groups disclosed in the present invention.

FIG. 2 shows an HPLC chromatogram at UV spectrum of CRC-023.

FIG. 3 shows results from the porcine coronary artery elution study comparing sirolimus, everolimus and CRC-023.

### DETAILED DESCRIPTION OF THE INVENTION

### Definitions

An important understanding of the mechanism of perivascular drug deposition from the perivascular space to the blood vessel wall has been advanced by the work of Lovich, Brown and Edelman. (Lovich et al., JACC, (29)7:1645-1650, 1997) These authors determined by utilizing in vivo animal studies with radiolabeled model drugs that, while a large portion of drug released into the perivascular space is cleared systemically through extravascular capillaries, most of the drug deposited into the vessel is supplied by direct inward diffusion from the source and little from systemic endovascular means.

A large variety of actual drug candidates for perivascular delivery have been investigated in both in vitro and in vivo studies. Concomitantly, investigative efforts to better understand the pathological basis of vascular remodeling following surgical procedures have provided guidance for drug selection. It is now recognized that endothelial changes associated with post-surgical vessel remodeling and possible resulting neointimal structural lesion (intimal hyperplasia (IH)) occurrence is central to both vein and arterial graft vessel narrowing (restenosis) and failure).

IH has been determined to originate from the abnormal migration and proliferation of vascular smooth muscle cells (SMC) with the deposition of extracellular connective tissue matrix resulting in restenosis. (Davies, et al., J Vasc Surg, (61)203-216, 2015) In the case of vascular grafting procedures, within hours, endothelial disruption by the surgical process initiates a mechanical and biochemical event cascade triggering SMC proliferation that continues for days or weeks with negative remodeling of the vessel. (Mitra, et al., Immunol Cell Biol, (84)115-112, 2006) It has been determined that resulting vascular graft restenotic lesions largely occur at the surgically connected regions. (Mills, et al., J Vasc Surg, (17)195-206, 1993)

Preclinical in vitro and model animal studies have shown that the compound sirolimus is effective in inhibiting vascular SMC proliferation. These investigations led to a large number of human studies utilizing sirolimus eluting cardiac stents that have demonstrated marked reduction of restenosis after stent implantation. (Marx, et al., Circ Cardiovasc Interv, (4)1:104-111, 2011) Oral delivery of sirolimus and everolimus (sirolimus derivative) has also shown SMC inhibiting activities, albeit with undesirable systemic side effects. (Kurdi, et al., Br J Clin Pharmacol, (82)5:1267-1279, 2015) Likewise sirolimus has also been shown using several human cancer cell lines to inhibit angiosarcoma at subtoxic doses. (Bundscherer, et al., Anticancer Res, (30)4017-4024, 2010)

These successes with sirolimus suggested and have resulted in several in vitro investigations utilizing perivascularly delivered sirolimus. All reported sirolimus perivascular studies have utilized an additional means to control drug migration and to extend drug delivery time to approximately 2 to 6 weeks. These means have included: wraps, gels and a variety of polymers, as well as micro- or nano-sized particles of varying chemical and mechanical complexities. Recently a mixture of sirolimus combined with hydrophobic cyanoacrylate(s) applied at the graft site has also been proposed as a means to retain the drug on target. (Tiansu-Chu, et al. Arq Bras Cardiol, (112)1:3-10, 2019) Presumably the increased hydrophobic and mechanical strength of the ensuing drug depot aids in this regard. This approach can also suffer from toxicity concerns as well as drug product manufacturing and end user complexities.

A sirolimus delivering collagen wrap (U.S. Pat. No. 6,726,923) is currently in U.S. clinical trials being conducted by Vascular Therapies, Inc. The use of wraps, however, suffer from requiring specialized surgical delivery requirements and can themselves induce negative remodeling. Material toxicities, administration complexities, manufacturing complexities, cost and regulatory status of these materials remain as issues to this approach.

The present invention provides for the synthesis of certain derivatives by addition of several cyclic hydrocarbon ester groups to the hydroxylethyl group on everolimus. These everolimus derivatives are shown below at Table 1. These compounds have proven to be extremely stable in vascular tissue and shows very slow elution from the tissue in a model study. Such behavior is extremely advantageous in preventing surgery induced intimal hyperplasia in “open” vasculature intervention.

### EXAMPLES

### Example Drug Molecules:

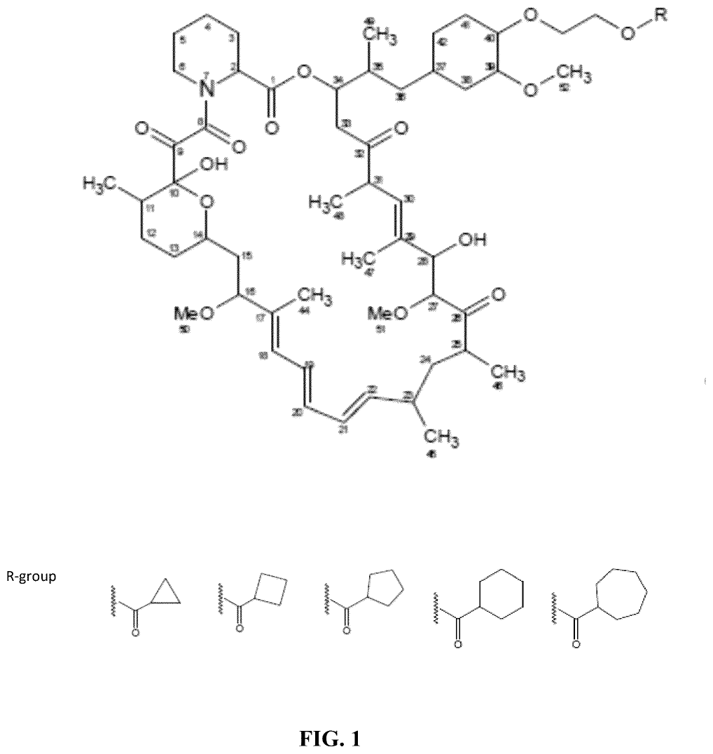

The macrocyclic triene immunosuppressive compounds of the present invention have more than one embodiment and may be described as comprising at least one of the following species from Table 1 and FIG. 1:

TABLE 1
Description of CRC-02X species

| | R Groups | Species |
|---|---|---|
| Main structure | H | Everolimus |
| 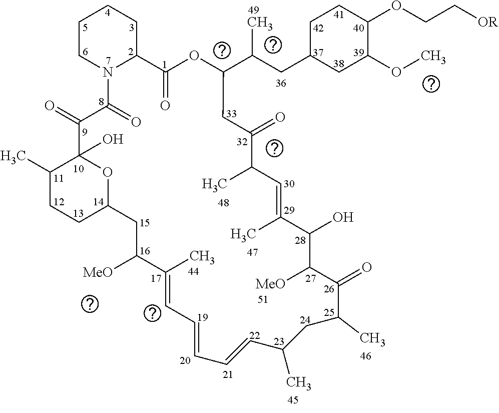 | 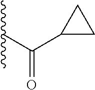 [SMILES: *C(=O)C1CC1] | CRC-023 a-e |
| | 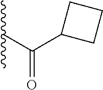 [SMILES: *C(=O)C1CCC1] | |
| | 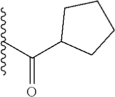 [SMILES: *C(=O)C1CCCC1] | |
| | 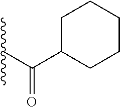 [SMILES: *C(=O)C1CCCCC1] | |
| | 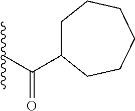 [SMILES: *C(=O)C1CCCCCC1] | |

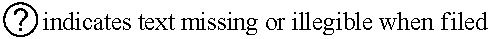

CRC-023 a-e are cyclopropane, cyclobutane, cyclopentane, cyclohexane, and cycloheptane ester derivatives of everolimus, respectively.

These compounds were synthesized and tested by RP-HPLC, UV, and high-resolution tandem mass spectrometry and results confirmed their respective molecular structures. Additionally, CRC-023 was synthesized, purified, and precipitated in milligram amounts yielding a white powder. CRC-023 was also characterized using aforementioned analytical techniques and results are consistent with the structure of everolimus cyclohexane ester.

### I. Chromatographic Conditions

HPLC analyses were performed using Agilent High Performance Liquid Chromatography (HPLC) 1290 Infinity Series coupled to a diode array detector G4212B (SN DEAA300117). The HPLC is equipped with a high performance autosampler G1329B (SN DEABE00184), thermostat G1330B (SN DEBAK00182), thermostatted column compartment G1316A (SN DEAAK00266), and quaternary pump G1311B (SN DEAAB00113). Data analysis was performed using Agilent Chemstation software for LC 3D systems.

LCMS analyses were performed using Agilent High Performance Liquid Chromatography (HPLC) 1290 Infinity Series coupled to a diode array detector G4212B (SN DEAA300734). The HPLC is equipped with an autoloop sampler G1367E (SN DEAAN00634), thermostat G1330B (SN DEBAK11543), thermostatic column compartment G1316C (DEBAC01381), degasser G1379B (JP60800486) and binary pump G1312B (SN DEAGD10783).

Mass spectrometry data was acquired using Agilent 6540A Quadrupole Time of Flight (QTOF) mass spectrometer (SN U.S. Pat. No. 93,980,202). Data analysis was performed using MassHunter Qualitative Analysis, Rev. B.04.00.

Table 2 shows HPLC and mass spectrometry parameters used for testing.

TABLE 2
HPLC and Mass Spectrometry Chromatographic Conditions
HPLC CONDITIONS

| Column | Supelco Ascentis Express C18 15 cm × | |
|---|---|---|
| | 4.6 mm, 2.7 μm | |
| Guard Column | Zorbax SB-C18 Guard Column, 4.6 × | |
| | 12.5 mm, 5 μm | |
| Detector Wavelength | 278 nm | |
| Detector Flow Cell | 10 mm | |
| Column Temperature | 45° C. | |
| Autosampler Temperature | 4° C. | |
| Flow Rate | 1.000 mL/min | |
| Injection Volume | Variable | |
| Mobile Phase | [Water (A)/1:1 Acetonitrile:Methanol | |
| | (B)] w/0.1% Formic Acid | |
| | Time (Minutes) | (A)/(B) |
| Gradient | 0 | 11.0/89.0 |
| | 12 | 11.0/89.0 |
| | 12.1 | 0.00/100.0 |

| | |
|---|---|
| Stop Time | 15 minutes |
| Post Time | 3 minutes |

MASS SPECTROMETER CONDITIONS

| | |
|---|---|
| Ion Source | Dual ESI |
| Ion Polarity | Positive |
| Collision Energy | Positive: 65 volts |
| Source Parameters | |
| Gas Temperature | 300° C. |
| Gas Flow | 13 L/min |
| Nebulizer | 60 psig |
| Scan Source Parameters | |
| VCap | 4000 |
| Fragmentor | 150 |
| Skimmer1 | 65 |
| Octopole RF Peak | 750 |

### II. Synthesis of Everolimus Cyclic Hydrocarbon Esters

Synthesis of various cyclic hydrocarbon ester derivatives of everolimus were performed according to the following procedure.

10 mg everolimus was weighed into a 2-mL HPLC glass vial. 50 μL dichloromethane was added into the vial and the solution was stirred at ambient temperature until everolimus was fully dissolved. 1.5 μL pyridine and 5 μL carbonyl chloride reactant (Table 3) were added into the solution.

The reaction mixture was stirred at ambient temperature for 10 minutes and then quenched by the addition of 1 mL ethyl acetate and 0.4 mL 1N hydrochloric acid. The organic phase was collected and washed with brine three times. The crude reaction was analyzed by HPLC and high-resolution mass spectrometry using chromatographic methods as detailed in Table 1.

TABLE 3
Reactants

| Compound | Carbonyl Chloride Reactant | Structure |
|---|---|---|
| Everolimus Cyclopropane Ester | Cyclopropanecarbonyl chloride | 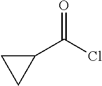 |
| Everolimus Cyclobutane Ester | Cyclobutanecarbonyl chloride | 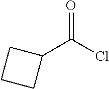 |
| Everolimus Cyclopentane Ester | Cyclopentanecarbonyl chloride | 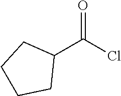 |
| Everolimus Cyclohexane Ester | Cyclohexanecarbonyl chloride | 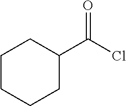 |
| Everolimus Cycloheptane Ester | Cycloheptanecarbonyl chloride | 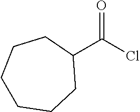 |

### III. Large Scale Synthesis and Purification of Everolimus Cyclohexane Ester (CRC-023)

200 mg everolimus was weighed into a 7-mL borosilicate glass vial. The drug was dissolved in 1 mL anhydrous tetrahydrofuran. 30 μL anhydrous pyridine and 100 μL cyclohexanecarbonyl chloride were added into the vial. A stir bar was placed into the vial and the reaction mixture was stirred at ambient temperature. After 25 minutes, the reaction mixture was transferred into a 125-mL separatory funnel. The 7-mL borosilicate glass vial reaction vessel was washed with a total of 10 mL ethyl acetate and the washes were added into the separatory funnel. 6 mL 1N HCl was added into the funnel. The solution was mixed by inverting the separatory funnel three times to mix the phases, venting the system after mixing. The aqueous phase was collected into a 30-mL beaker and back-extracted with 5 mL ethyl acetate twice. The organic layers were pooled together and washed with 30 mL brine four times until an approximate pH of 5.5-6.5 by litmus paper was reached.

Excess water was removed from the organic phase by the addition of sodium sulfate until the added solid was freely mobile at the bottom of the beaker after stirring with a stirring rod. The solution was decanted into a 100-mL round bottom flask and concentrated under vacuum via rotovap with water bath maintained at 37° C.

### Purification by Open Column Chromatography

An open glass column (1.75×12 in) was assembled and packed with 100 g silica and 500 mL solution of 40/60 ethyl acetate/n-heptane (v/v). Method parameters for purification are listed in Table 4.

TABLE 4
Open Column Chromatography Method Parameters
Flash Chromatography Parameters

| | |
|---|---|
| Column | 1.75 × 12 inches |
| Silica Gel | 200-400 mesh, 60 Å, 100 g |
| Flow Rate | 6 mL/min |
| Mobile Phase | 40/60 Ethyl Acetate/n-Heptane (v/v) |
| Gradient | Isocratic |
| Fraction Test Tube Size | 16 × 125 mm |
| Fraction Volume | Approximately 15 mL |

The crude product in the 100-mL round bottom flask was loaded carefully onto the column with a pipette making sure not to disturb the column bed. The round bottom flask was rinsed three times with approximately 0.5 mL mobile phase and the washes were also loaded onto the column. The column stopcock was opened to allow the sample to enter the column bed. The mobile phase was then carefully added onto the column and the column stopcock was opened to begin purification. Approximately 360 mL void volume was collected followed by collection of fractions containing approximately 15 mL of solvent. The presence of product was monitored by TLC using 60/40 ethyl acetate/hexanes mobile phase. CRC-023 has a TLC Rf value of approximately 0.5.

Various fractions containing CRC-023 by TLC were tested by HPLC to verify product purity and approximate compound amounts using everolimus as the standard. Fractions with CRC-023 chromatographic area percent of >97% were combined into a 1-L round bottom flask. The solution was concentrated under vacuum via rotovap with water bath maintained at 37° C.

### CRC-023 Methanol Precipitation

Assuming 150 mg of CRC-023 was recovered from purification, the sample from the above section was reconstituted in 420 μL methanol. 3.9 mL ice cold HPLC grade water was transferred into a 7-mL borosilicate glass vial with stir bar and the vial was placed into an ice bath. Using a 100-μL syringe, the sample dissolved in methanol was slowly added dropwise into the vial of water with vigorous stirring. The precipitated product in water was filtered through a 15-mL medium porosity sintered glass funnel under vacuum. The white solid product was quantitatively transferred into a 4-mL borosilicate glass vial using a spatula. The vial was covered with aluminum foil with multiple 1 mm punctured holes.

The vial was placed into the oven for drying at 45° C. under vacuum at pressure of −28.0 inches Hg. After a 24-hour drying period, CRC-023 was removed from the oven and allowed to reach ambient temperature. A 200 μg/mL solution of the final product was prepared in acetonitrile and tested by HPLC and high-resolution mass spectrometry using conditions detailed in Table 2. CRC-023 was stored under desiccation at ≤−78° C.

### IV. Results of HPLC and Mass Spectrometry Analyses of Everolimus Cyclic Hydrocarbon Esters

The molecular structures of everolimus cyclic hydrocarbon esters synthesized in accordance with the above descriptions are shown at FIG. 1: cyclopropane, cyclobutane, cyclopentane, cyclohexane, and cycloheptane.

The results of mass spectrometry analyses of everolimus cyclic hydrocarbon esters are shown in Table 5. The molecular mass [M+Na]+ detected for each compound matched the molecular formula of that material. The detected masses are also within the recommended ±0.003 m/z units of calculated masses considered adequate for supporting a molecular formula for compounds.

TABLE 5
Mass Spectrometry Results of Everolimus Cyclic Hydrocarbon Esters

| | | | Calculated | Found | Mass |
|---|---|---|---|---|---|
| | Species | Molecular | Monoisotopic | Monoisotopic | Difference |
| Compound | Name | Formula | Mass [M + Na]^{+ [4]} | Mass [M + Na]⁺ | (m/z)* |
| Cyclopropane Ester | CRC-023a | C₅₇H₈₇NO₁₅Na⁺ | 1048.5973 | 1048.5987 | −0.001 |
| Cyclobutane Ester | CRC-023b | C₅₈H₈₉NO₁₅Na⁺ | 1062.6130 | 1062.6161 | −0.003 |
| Cyclopentane Ester | CRC-023c | C₅₉H₉₁NO₁₅Na⁺ | 1076.6286 | 1076.6276 | 0.001 |
| Cyclohexane Ester | CRC-023d | C₆₀H₉₃NO₁₅Na⁺ | 1090.6443 | 1090.6441 | 0.000 |
| Cycloheptane Ester | CRC-023e | C₆₁H₉₅NO₁₅Na⁺ | 1104.6600 | 1104.6602 | 0.000 |

*Found masses are within the recommended ±0.003 m/z units of calculated values considered adequate for supporting a molecular formula for compounds.

The mass fragments detected for everolimus cyclic hydrocarbon esters are shown in Table 6. Also shown are mass fragments of everolimus for comparison. Mass fragments 320, 381, 409, 441, 453, 485, 582, and 614 m/z are fragments that do not contain the region of the molecule being modified, hence, these fragments are detected for everolimus and its cyclic hydrocarbon ester derivatives. Everolimus fragments 389, 651, 686, and 775 m/z contain the site of the molecule where modification occurs for CRC-023 a-e. These fragments were absent in the MS/MS spectra of cyclic hydrocarbon ester derivatives of everolimus and replaced by respective mass fragments unique to each compound as detailed in Table 6. Results of tandem mass spectrometry analyses confirmed the molecular structures of these compounds.

TABLE 6
MS/MS Results of Everolimus Cyclic Hydrocarbon Esters

| | | Molecular | | Detected |
|---|---|---|---|---|
| | Molecular | Ion | Detected | Fragments Unique |
| Compound | Formula | [M + Na]⁺ | Fragments | to Compound |
| Everolimus | C₅₃H₈₃NO₁₄Na⁺ | 980.5711 | 320, 381, 409, 441, | 389, 651, 686, 775 |
| | | | 453, 485, 582, 614 | |
| Cyclopropane Ester | C₅₇H₈₇NO₁₅Na⁺ | 1048.5973 | 320, 381, 409, 441, | 457, 719, 754, 843 |
| (CRC-023a) | | | 453, 485, 582, 614 | |
| Cyclobutane Ester | C₅₈H₈₉NO₁₅Na⁺ | 1062.6130 | 320, 381, 409, 441, | 471, 733, 768, 857 |
| (CRC-023b) | | | 453, 485, 582, 614 | |
| Cyclopentane Ester | C₅₉H₉₁NO₁₅Na⁺ | 1076.6286 | 320, 381, 409, 441, | 485, 747, 782, 871 |
| (CRC-023c) | | | 453, 485, 582, 614 | |
| Cyclohexane Ester | C₆₀H₉₃NO₁₅Na⁺ | 1090.6443 | 320, 381, 409, 441, | 499, 761, 796, 885 |
| (CRC-023d) | | | 453, 485, 582, 614 | |
| Cycloheptane Ester | C₆₁H₉₅NO₁₅Na⁺ | 1104.6600 | 320, 381, 409, 441, | 513, 775, 810, 899 |
| (CRC-023e) | | | 453, 485, 582, 614 | |

### V. Results of Large-Scale Synthesis and Purification of Everolimus Cyclohexane Ester (CRC-023)

Synthesis, purification, and methanol precipitation of CRC-023 was completed obtaining 133 mg of white powder. FIG. 2 shows the RP-HPLC chromatogram of CRC-023 at 278 nm showing an elution time of 6.5 minutes for CRC-023 with area percent of 97.8%.

The crude reaction of everolimus with respective cyclic carbonyl chloride reactants yielded cyclopropane (CRC-023a), cyclobutane (CRC-023b), cyclopentane (CRC-023c), cyclohexane (CRC-023d), and cycloheptane (CRC-023e) ester derivatives of everolimus. These compounds have RPHPLC retention times greater than everolimus suggesting increased lipophilicity. The retention times of these compounds were also observed to increase with every addition of one carbon comprising the cyclic ring from CRC-023 a-e. The UV spectrum of CRC-023 a-e showed maximum absorptions at 268, 278, and 288 nm corresponding to the conjugated triene moiety that is unique to sirolimus and its derivatives.

Results of MS and MS/MS analyses of these compounds confirmed their respective molecular formulas and molecular structures. Large scale (200 mg) synthesis, purification, and methanol precipitation of CRC-023 has been performed to yield 133 mg white powder. This material was characterized by RP-HPLC, UV, MS, and MS/MS. CRC-023 was found to have an HPLC area percent of 98.7%.

UV analysis of this material showed a conjugated triene with maximum absorptions at 268, 278, and 288 nm. The molecular mass and mass fragments detected from testing of this compound by high resolution tandem mass spectrometry are consistent with the structure of CRC-023 confirming its identity to be the cyclohexane ester derivative of everolimus.

In conclusion, the cyclopropane, cyclobutane, cyclopentane, cyclohexane, and cycloheptane ester derivatives of everolimus, as described in the present invention, were synthesized and tested by RP-HPLC, UV, and high-resolution tandem mass spectrometry and results confirmed their respective molecular structures. Additionally, CRC-023 was synthesized, purified, and precipitated in milligram amounts yielding a white powder. CRC-023 was also characterized using aforementioned analytical techniques and results are consistent with the structure of everolimus cyclohexane ester.

### VI. Elution Procedures on Porcine Coronary Artery

To examine the effects of certain formulations of the present invention on porcine coronary artery samples, the samples were each prepared as follows:

Sirolimus Formulation Preparation: Sirolimus was weighed, 3.118 mg, into a 2-mL HPLC vial. Ethanol (200 proof) was added to the vial, 98.40 mg (125 μL), using a 1000 μL Eppendorf® pipette to give a final concentration of 24.9 mg/mL.

Everolimus Formulation Preparation: Everolimus was weighed, 3.141 mg, into a 2-mL HPLC vial. Ethanol (200 proof) was added to the vial, 99.021 mg (126 μL), using a 1000 μL Eppendorf® pipette to give a final concentration of 24.9 mg/mL.

CRC-023 Formulation Preparation: CRC-023 was weighed, 3.223 mg, into a 2-mL HPLC vial. Ethanol (200 proof) was added to the vial, 99.753 mg (126 μL), using a 1000 μL Eppendorf® pipette to give a final concentration of 25.5 mg/mL.

Elution Media Preparation: Normal saline was prepared by weighing 4.5 grams of sodium chloride into a 500 mL Pyrex® bottle. 495.5 grams HPLC water was added to the 500 ml bottle. The bottle was then capped and vigorously shaken. 25% ethanol/saline elution media was prepared by weighing 25.0 grams of ethanol (200 proof) into a 100 mL Pyrex® bottle. 75.0 grams of normal saline was added to the 100 ml bottle containing ethanol. The bottle was capped and vigorously shaken.

Elution Method: Porcine coronary arterial vessels were obtained and dissected from hearts obtained from the butcher shop. Coronary arteries were flash frozen with dry ice then stored at −80° C. When ready for testing, arteries were removed from the freezer and allowed to thaw at room temperature. Any fat and muscle tissue were carefully removed using scissors and tweezers. Vessels were rinsed several times with normal saline containing 0.05% sodium azide.

Vessels were then cut into 1 cm sections. 5 μL of formulation (approximately 125 μg of drug) was carefully added onto the outer part of the tissue using a 10 μL Hamilton® syringe. Ethanol was allowed to evaporate at room temperature for 15-20 minutes.

Tissue was then placed into a 7 mL glass vial. 2 mL elution media, previously warmed to 37° C., was added carefully to each vial using an Eppendorf® pipette. Each vial was capped and placed in a 37° C. incubator and gently shaken at 100 spm (strokes per minute).

At certain time points, elution media was removed and fresh 2 mL media (at 37° C.) was slowly added to the vial.

VII. Results from Elution Study on Porcine Coronary Artery Comparing Sirolimus, Everolimus and CRC-023

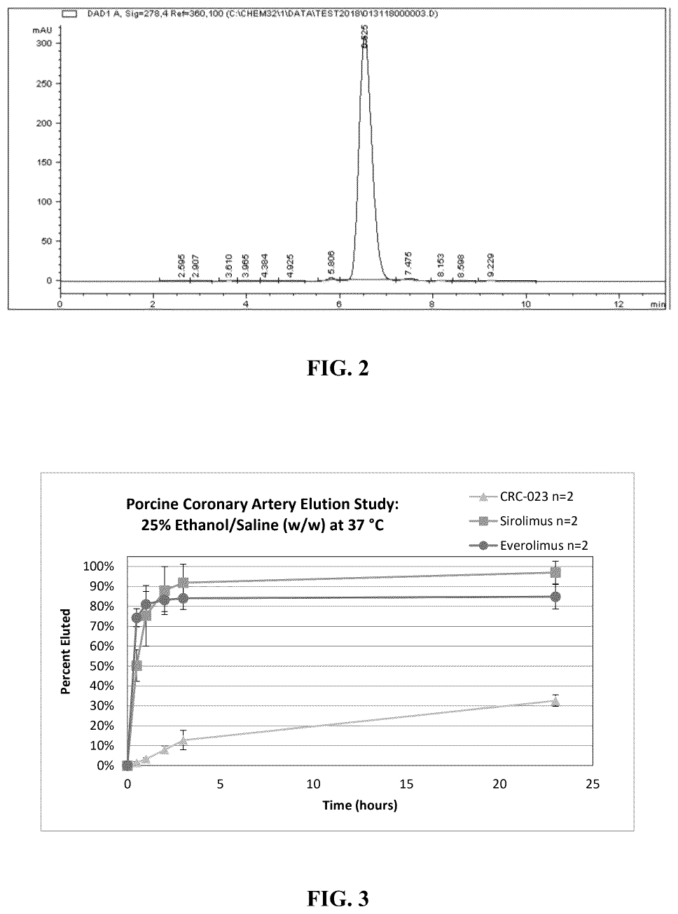

Elution studies were performed on porcine coronary arterial tissue with sirolimus, everolimus and CRC-023. Drug formulations in ethanol were applied (approximately 125 μg) onto previously frozen “butcher shop” porcine coronary arterial vessel and allowed to dry after 15-20 minutes. An elution study was then performed as described in Example VI above. Results are shown in FIG. 3 and Table 7.

TABLE 8
Percent of Drug Eluted Over Time from Elution Study

| | | | |
|---|---|---|---|
| Time | CRC-023 | Sirolimus | Everolimus |

| (hours) | 1 | 2 | 1 | 2 | 1 | 2 |
|---|---|---|---|---|---|---|
| 0.5 | 1% | 2% | 56% | 45% | 77% | 71% |
| 1 | 3% | 4% | 86% | 65% | 86% | 76% |
| 2 | 9% | 7% | 96% | 79% | 87% | 79% |
| 3 | 16% | 9% | 98% | 85% | 88% | 80% |
| 23 | 35% | 31% | 101% | 93% | 89% | 80% |
| Extract | 68% | 65% | 102% | 94% | 89% | 81% |

The results clearly show sirolimus and everolimus eluting significantly faster than CRC-023, with most of the drug eluting off the tissue within the first 2 hours of the study. On the contrary, CRC-023 slowly eluted, with most of the drug recovered after extraction. This is a clear indication that not only the rapamycin 40-O-cyclic hydrocarbon ester compounds show very favorable elution behavior for perivascular application, but also exceptional stability.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of ordinary skill in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

What is claimed is:

1. A rapamycin 40-O-cyclic hydrocarbon ester compound having the structure: 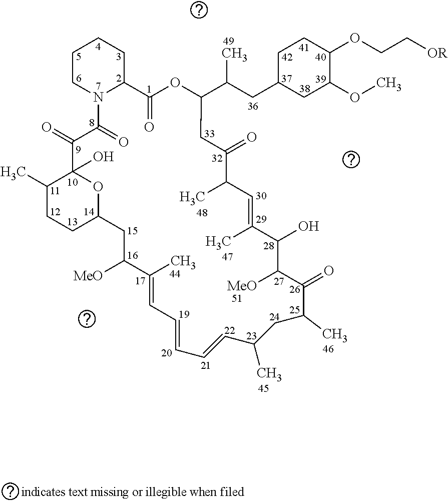
where R is a saturated cyclic hydrocarbon substituent.

2. The rapamycin 40-O-cyclic hydrocarbon ester compound of claim 1, wherein R is one of the following structures:

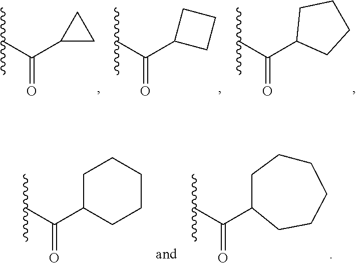

3. A method of treating open vascular surgical manipulations comprising administering an effective amount of the compound according to claim 1 by direct perivascular application.

4. The method of claim 3, wherein the compound is formulated to allow for direct perivascular application directly to graft vessels upon initiation of surgery.

5. The method according to claim 3 or 4, wherein the compound is stabile when administered to a coronary artery.

6. The method according to claim 3 or 4, wherein the compound is stabile when administered to a peripheral artery.

7. The method according to any of claims 3 to 5, wherein the compound, once administered, results in superior vessel retention and reduced tissue diffusion.

8. A formulation in particular for treating a vascular injury comprising
rapamycin 40-O-cyclic hydrocarbon ester compound having the structure: 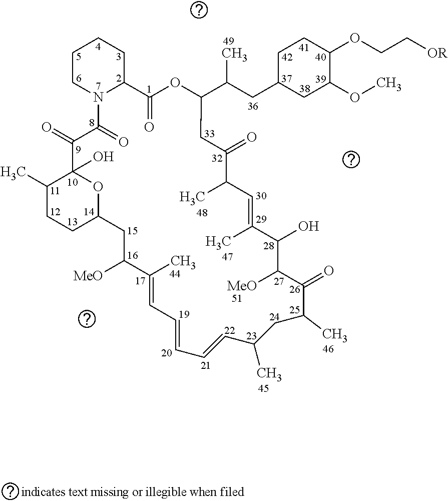
where R is a saturated cyclic hydrocarbon substituent, wherein the formulation further comprises at least one solvent, wherein the solvent consists of or comprises at least one solubilizer or solubilizer mixture, wherein the solubilizer can be selected from the group consisting of alcohols such as ethanol, propanol, butanol, benzyl alcohol, glycerol, polytheylene glycol and propylene glycol, dimethyl sulfoxide, dimethylformamide, propylene benzyl benzoate, glycol monolaurate, Labrasol®, Kolliphor®, and amylene hydrate.

9. The formulation of claim 8, wherein the at least one solubilizer is ethanol.

10. An implant comprising an effective amount of the rapamycin 40-O-cyclic hydrocarbon ester compound of claim 1.

11. The implant of claim 10, wherein the implant is selected from the group consisting of patches, wound patches, suture materials, grafts.
